(19) United States
(12) Patent Application Publication
(10) Pub. No.: US 2026/0294570 A1
(43) Pub. Date: Oct. 1, 2026

(54) RADIOTHERAPY PATIENT POSITIONING SYSTEM PROTECTIVE COVER

(71) Applicant: Varian Medical Systems, Inc., Palo Alto, CA (US)

(72) Inventors: Johannes STOCK, Reuth bei Erbendorf (DE); Klaus HRUSCHKA, Erbendorf (DE); Berthold BAUMANN, Kastl (DE); Daniel EIGNER, Erbendorf (DE); Patrick PLANNERER, Kulmain (DE); Jochen Dieter HECHT, Dachsbach (DE); Viola SUGAYA, Wettingen (CH); Katrin KILIAN, Weiden (DE)

(73) Assignee: Varian Medical Systems, Inc., Palo Alto, CA (US)

(21) Appl. No.: 19/629,179

(22) Filed: Mar. 26, 2026

(30) Foreign Application Priority Data
Mar. 27, 2025 (EP) 25166696.2

(51) Int. Cl. A61B 46/10

(52) U.S. Cl. CPC A61B 46/10

(57) ABSTRACT

A protective cover for a radiotherapy patient positioning system is disclosed. The radiotherapy patient positioning system has at least two parts that are moveable relative to one another. The protective cover comprises at least a first cover section that is configured to cover a first part of the at least two parts and to overhang a second part of the at least two parts of the patient positioning system. The protective cover may include a second cover section that is configured to cover the second part, wherein the first cover section is configured to overhang the second cover section. A size and a shape of the first cover section and a size and a shaped of the second cover section are preferably such that liquid running down the protective cover is guided away from the at least two parts.

### CROSS-REFERENCE TO RELATED APPLICATION(S)

The present application claims priority under 35 U.S.C. § 119 to European Patent Application No. 25166696.2, filed Mar. 27, 2025, the entire contents of which is incorporated herein by reference.

### TECHNICAL FIELD

One or more example embodiments relates to a protective cover for a radiotherapy patient positioning system that has at least two parts that are moveable relative to one another. One or more example embodiments also relates to a radiotherapy patient positioning system covered with the protective cover.

### RELATED ART

Radiation therapy is a localised treatment for a specific target tissue (a planning target volume), such as a cancerous tumour. Ideally, radiation therapy is performed on the planning target volume that spares the surrounding normal tissue from receiving doses above specified tolerances, thereby minimising risk of damage to healthy tissue. Prior to the delivery of radiation therapy, an imaging system is typically employed to provide a three-dimensional image of the target tissue and surrounding area. From such imaging, the size and mass of the target tissue can be estimated and an appropriate treatment plan generated and planning target volume determined.

So that the prescribed dose is correctly supplied to the planning target volume (i.e., the target tissue) during radiation therapy, the patient should be correctly positioned relative to the linear accelerator that provides the radiation therapy. Typically, dosimetric and geometric data are checked before and during the treatment, to ensure correct patient placement and that the administered radiotherapy treatment matches the previously planned treatment. This process is referred to as image guided radiation therapy (IGRT), and involves the use of an imaging system to view target tissues while radiation treatment is delivered to the planning target volume. IGRT incorporates imaging coordinates from the treatment plan to ensure the patient is properly aligned for treatment in the radiation therapy device.

A radiotherapy patient positioning system is used to support and position the patient during treatment and/or imaging. A radiotherapy patient positioning system typically includes a couch that supports the patient in a supine position. The radiotherapy patient positioning system typically further includes a mechanism for moving the couch (and the supported patient).

The movement between different moveable parts of a radiotherapy patient positioning system result in a device that has a changeable size and shape. The movement between different moveable parts of a radiotherapy patient positioning system can make it difficult to keep the radiotherapy patient positioning system clean and to prevent the ingress of fluids (for example, from the patient).

A radiotherapy patient positioning system may have six axes of movement. Providing the six axes is a particular challenge, as the complex mobility of the axes is characterised by the high number of degrees of freedom. To ensure the functionality of the radiotherapy patient positioning system, the degrees of freedom must be strictly adhered to in order to avoid collisions between the static and moving parts. This creates an increased risk of pinch points, especially at the transitions between the axles and other moving components, which poses both safety concerns and technical challenges.

Another problem is the cleanability of the components. The large number of movements and axes makes it difficult to access certain areas, which makes thorough cleaning complicated. This can be a serious problem in environments where cleanliness plays a crucial role, such as therapy systems.

In addition, there is a risk that hoses, cables or other accessories that move with the axes could become trapped or damaged during operation. This requires special precautions when guiding lines and selecting accessories to ensure that they can follow the movements of the axes without being blocked or damaged. Appropriate gap dimensions must also be observed in accordance with the IEC.

It has been proposed to cover a vertical section of a radiotherapy patient positioning system, which provides heigh adjustment, with bellows—which help to protect mechanical components by providing flexible covers. These bellows are often made of elastomers, which are known for their flexibility and elasticity. However, elastomers can become brittle over time, especially when exposed to extreme temperatures, UV radiation or chemical stresses. Brittle bellows lose their protective function and can easily crack or break, leaving the underlying components unprotected. This increases the risk of contamination, mechanical wear or damage caused by external influences. In addition, the production of bellows is complex and must be secured against pressure accordingly so that there is no unintentional penetration into moving mechanics.

### SUMMARY

In one aspect, one or more example embodiments provides a protective cover for a radiotherapy patient positioning system having at least two parts that are moveable relative to one another, the protective cover comprising at least a first cover section that is configured to cover a first of said parts and to overhang a second of said parts.

By the first cover section being configured to overhang the second of said parts, the first cover section may divert liquid away from the second of said parts to reduce the likelihood that liquid flowing down the first cover section will enter the second of said parts and potentially cause damage.

The protective cover may include a second cover section that is configured to cover the second of said parts, wherein the first cover section is configured to overhang the second cover section. The second cover section may protect the second of said parts. By the first cover section being configured to overhang the second cover section, the first cover section may divert liquid away from any gap between the first and second cover sections to reduce the likelihood that liquid flowing down the first cover section will enter the second of said parts and potentially cause damage.

The size and shape of the first cover section and the second cover section may be such that liquid running down the protective cover is guided away from the parts of radiotherapy patient positioning system covered by the first cover section and the second cover section.

The first cover section may be configured to partially overlap the second cover section. By the first cover section being configured to partially overlap the second cover section this may reduce the likelihood that liquid flowing down the first cover section will enter the second of said parts and potentially cause damage and may reduce the likelihood of any components getting caught between the first and second cover sections

The first cover section may be slidably coupled to the second cover section to accommodate movement between the first of said parts and the second of said parts of the radiotherapy patient positioning system.

The first cover section and the second cover section may comprise a telescopic side wall structure, wherein the telescopic side wall structure is shaped to fit around the two parts of the positioning system that are moveable relative to one another and the telescopic side wall structure is configured to expand or contract with relative movement of the two parts of the positioning system. Preferably, the first cover section may have a first side wall structure that overlaps a second side wall structure of the second cover section by a variable amount to accommodate relative movement of the two parts of the positioning system, wherein a lower edge of the first side wall structure includes an inwardly extending flange and an upper edge of the second side wall structure includes a outwardly extending flange, the inwardly extending flange and the outwardly extending flange being configured to engage one another to prevent the first side wall structure and the second side wall structure separating in response to the relative movement of the two parts of the positioning system. Such first and second cover sections may form a vertical support cover section in the embodiment to be described.

The first cover section may include at least one lip that is configured to overhang the second of said parts. The at least one lip may be configured to partially overlap the second of said parts. The at least one lip may extend in a first direction along a side of the first cover section and may be configured to allow movement of the first of said parts relative to the second of said parts in the first direction. Such a first cover section may form a couch cover section in the embodiment to be described. The second of the parts may be covered by a second cover section.

The first cover section may extend generally vertically, the protective cover comprising a further cover section that extends generally horizontally, the further cover section being provided with one or more drainage holes. Such a further cover section may form part of a longitudinal coupling cover section in the embodiment to be described.

The first cover section may extend generally vertically, the protective cover comprising a further cover section that extends generally horizontally, the further cover section being provided with one or more protrusions for guiding flow of liquid in a desired direction. Such a further cover section may form part of a foot cover section in the embodiment to be described.

The sections of the cover may, for example, be one of, or a part of, any of the following:

A footrest cover section that is configured to cover a footrest of the radiotherapy patient positioning system.

A pitch roll cover section that is configured to cover a pitch roll coupling of the radiotherapy patient positioning system.

A longitudinal coupling cover section that is configured to cover a longitudinal coupling of the radiotherapy patient positioning system.

A vertical support cover section that is configured to cover a vertical support of the radiotherapy patient positioning system.

One of several cover parts comprising the vertical support cover section.

A foot cover section that is configured to cover a foot of the radiotherapy patient positioning system.

A base cover section that is configured to cover a base of the radiotherapy patient positioning system.

A yaw module cover section that is configured to cover a yaw module of the radiotherapy patient positioning system.

One or more example embodiments provides a radiotherapy patient positioning system covered with the protective cover as specified above.

A radiotherapy patient positioning apparatus according to one or more example embodiments includes:
a radiotherapy patient positioning system having at least two parts that are moveable relative to one another; and
a protective cover comprising at least a first cover section arranged to cover a first of said parts and to overhang a second of said parts.

A second cover section may be arranged to cover the second of said parts, wherein the first cover section overhangs the second cover section.

The size and shape of the first cover section and the second cover section may be such that liquid running down the protective cover is guided away from the parts of radiotherapy patient positioning system covered by the first cover section and the second cover section.

The first cover section may partially overlap the second cover section.

The first cover section may be slidably coupled to the second cover section to accommodate movement between the first of said parts and the second of said parts of the radiotherapy patient positioning system.

The first cover section and the second cover section may comprise a telescopic side wall structure, wherein the telescopic side wall structure is shaped to fit around the two parts of the positioning system that are moveable relative to one another and the telescopic side wall structure is configured to expand or contract with relative movement of the two parts of the positioning system.

The first cover section may have a first side wall structure that overlaps a second side wall structure of the second cover section by a variable amount to accommodate relative movement of the two parts of the positioning system, wherein a lower edge of the first side wall structure includes an inwardly extending flange and an upper edge of the second side wall structure includes a outwardly extending flange, the inwardly extending flange and the outwardly extending flange being configured to engage one another to prevent the first side wall structure and the second side wall structure separating in response to the relative movement of the two parts of the positioning system.

The first cover section may include at least one lip that overhangs the second of said parts.

The at least one lip may partially overlap the second of said parts.

The at least one lip may extend in a first direction along a side of the first cover section and may be configured to allow movement of the first of said parts relative to the second of said parts in the first direction.

The size and shape of the first cover section may be such that liquid running down the protective cover is guided away from the second of said parts of radiotherapy patient positioning system.

The first cover section may extend generally vertically, the protective cover comprising a further cover section that extends generally horizontally, the further cover section being provided with one or more drainage holes.

The first cover section may extend generally vertically, the protective cover comprising a further cover section that extends generally horizontally, the further cover section being provided with one or more protrusions for guiding flow of liquid in a desired direction.

The overlapping cover design from top to bottom of the cover may be based on the so-called roof tile principle, where the covers or protective layers are arranged in such a way that they overlap and form a seamless barrier. Similar to roof tiles, each top cover is placed over the one below to prevent liquids, dust, or other foreign objects from entering. This may create an effective protective layer that is particularly useful in applications where sensitive or moving parts need to be protected from external influences.

The nesting of the covers takes place from the inside to the outside, so that no (or little) liquid or contamination may get to the load-bearing or moving, driven parts of the radiotherapy treatment system. This arrangement may be particularly beneficial in applications where liquids such as urine, water or detergents are used, as it provides that they tend not to penetrate into areas where they could cause damage or malfunction. The overlapping and nesting keeps the mechanics underneath protected, increasing the longevity and reliability of the entire system.

In summary, this design may offer robust protection against external influences due to its overlapping and nesting and is ideal for applications where moving and driven parts are particularly sensitive to liquids and dirt.

Embodiments may provide:
Simplified assembly due to reduced component scope, giving a cost advantage
Integrated interfaces on a highly integrated component in the assembly for e.g. exact movement of the patient along an axis
Complex geometries in the product can be implemented

### BRIEF DESCRIPTION OF DRAWINGS

The accompanying drawings, which are incorporated in and form a part of this specification and in which like numerals depict like elements, illustrate embodiments of the present disclosure and, together with the detailed description, serve to explain the principles of the disclosure.

FIG. 1 is a schematic perspective view of a radiation treatment system.

i. FIG. 2 is front elevation of a radiotherapy patient positioning system indicating the position of parts of the system.

ii. FIG. 3 is a perspective front and right side view of the radiotherapy patient positioning system including a cover (left side corresponds).

iii. FIG. 4 is front elevation of the radiotherapy patient positioning system including the cover.

iv. FIG. 5 is a perspective rear and right side view of the radiotherapy patient positioning system including the cover.

v. FIG. 6 is an enlarged partial front elevation of the radiotherapy patient positioning system including the cover, showing a footrest cover section, a pitch roll cover section and a longitudinal coupling cover section.

vi. FIG. 7 is an enlarged partial vertical cross-section of the radiotherapy patient positioning system including the cover, showing part of the footrest cover section, the pitch roll cover section and part of the longitudinal coupling cover section.

vii. FIG. 8 is an enlarged partial vertical cross-section of the radiotherapy patient positioning system including the cover, showing part of the pitch roll cover section and the longitudinal coupling cover section.

viii. FIG. 9 is an enlarged partial vertical cross-section of the radiotherapy patient positioning system including the cover, showing part of the longitudinal coupling cover section, a vertical support cover section and part of a foot cover section.

ix. FIG. 10 is an enlarged partial vertical cross-section of the radiotherapy patient positioning system including the cover, showing part of the vertical support cover section, the foot cover section and part of a base cover section.

x. FIG. 11 is an enlarged partial perspective front and left side view of part of the vertical support cover section, the foot cover section and the base cover section.

### DETAILED DESCRIPTION

Reference will now be made in detail to the various embodiments of the present disclosure, examples of which are illustrated in the accompanying drawings. While described in conjunction with these embodiments, it will be understood that they are not intended to limit the disclosure to these embodiments. On the contrary, the disclosure is intended to cover alternatives, modifications and equivalents, which may be included within the scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

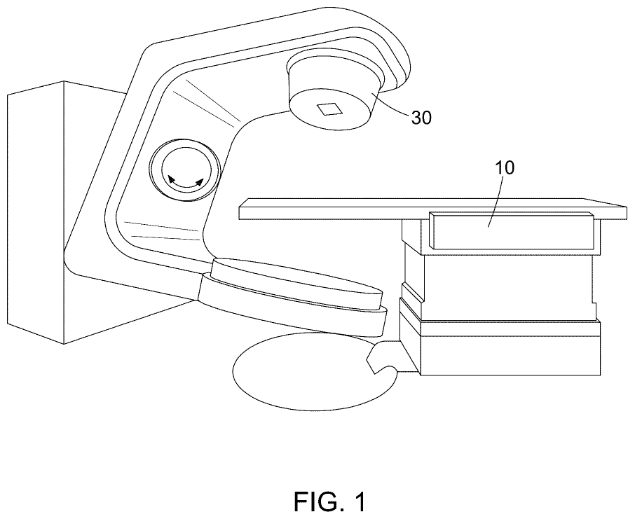

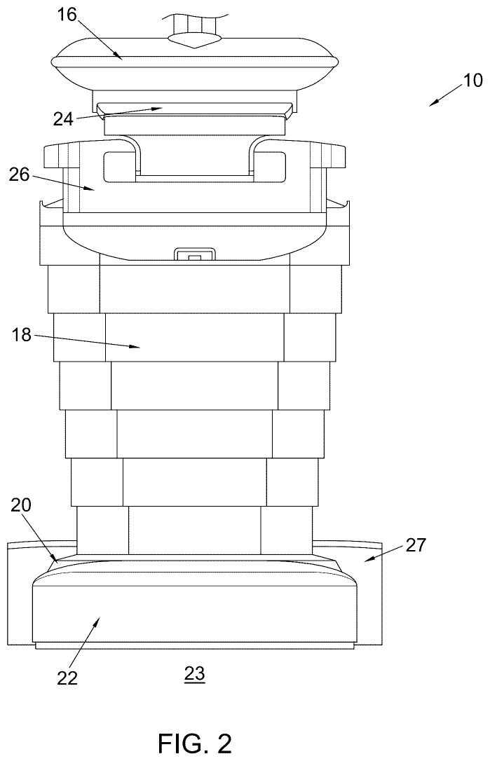

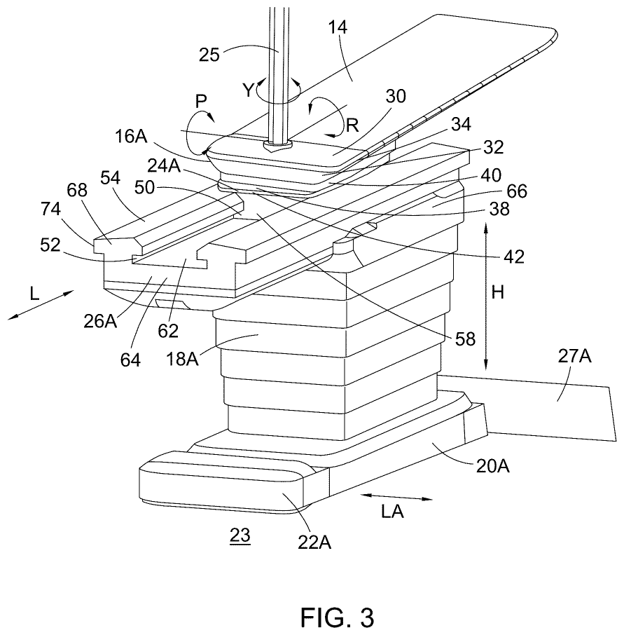

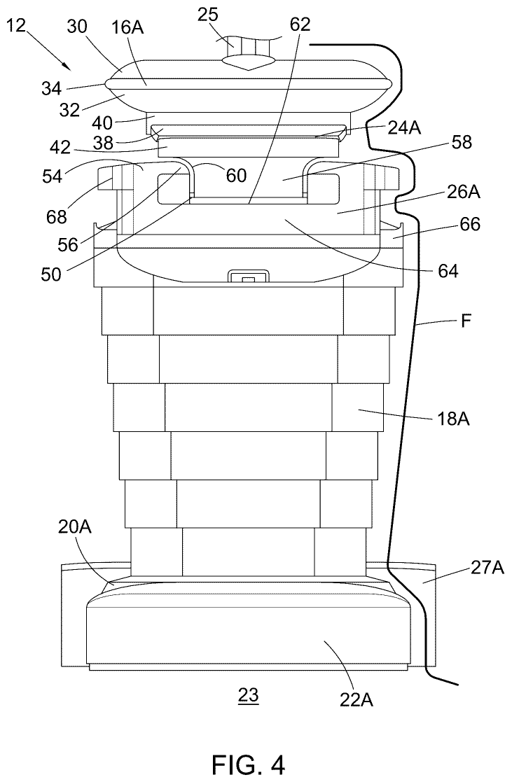

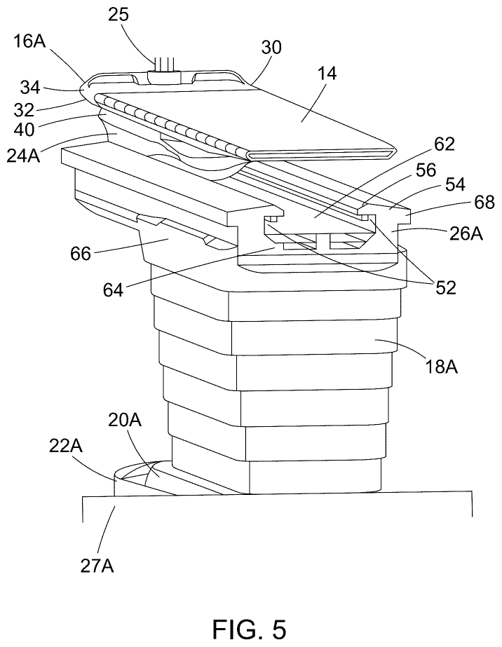

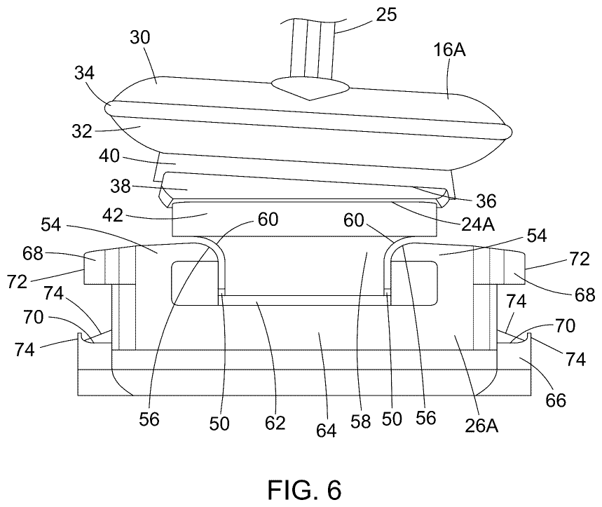

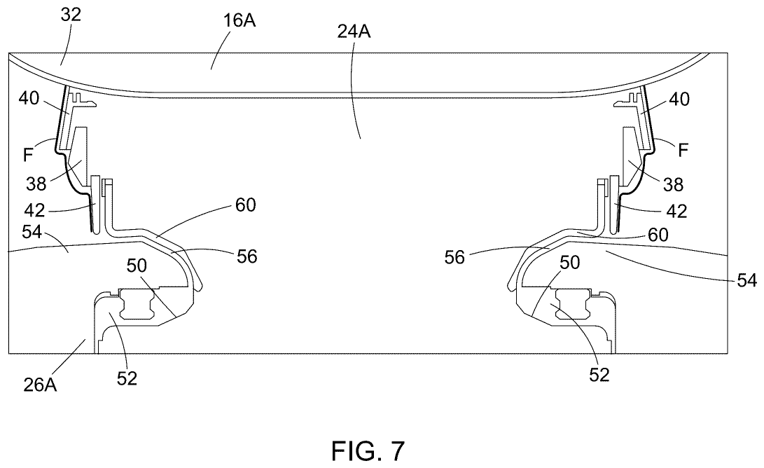

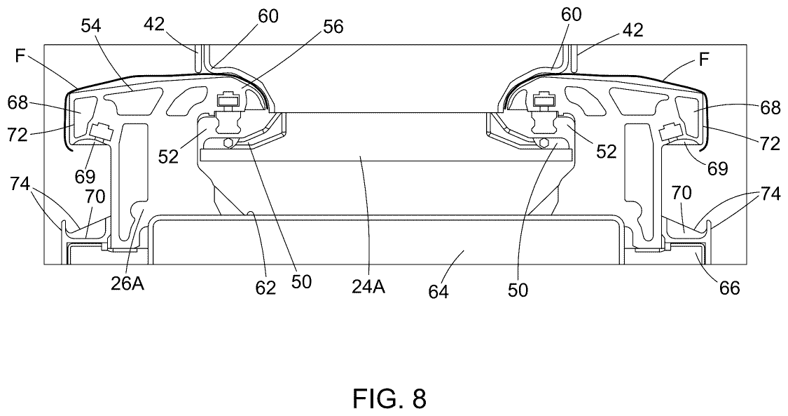

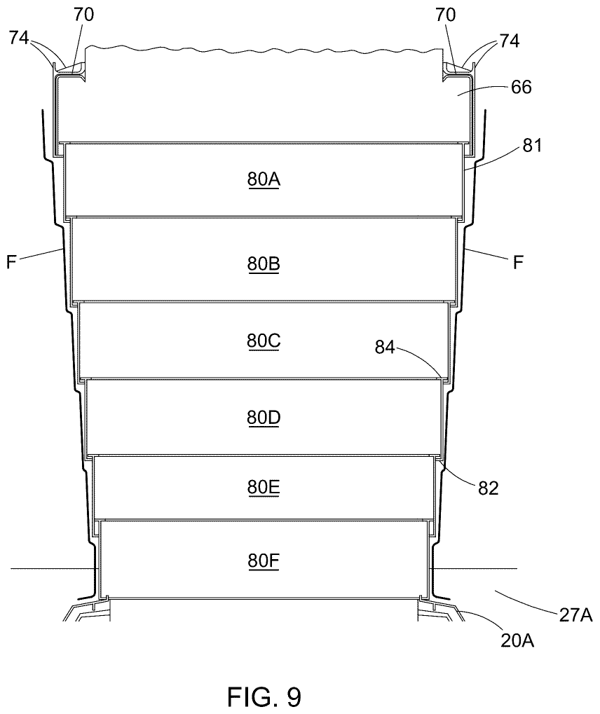

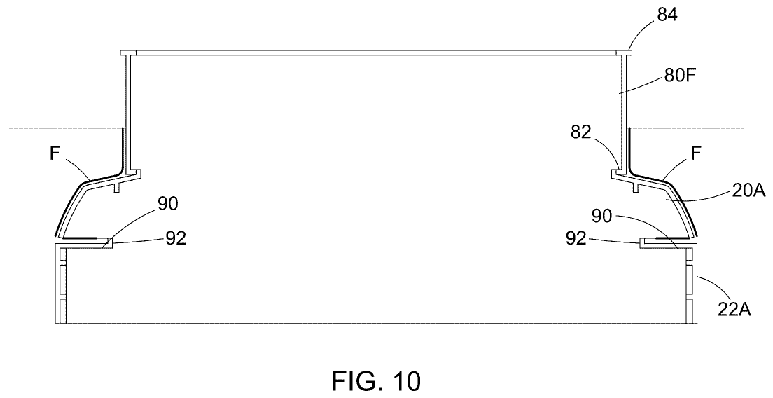

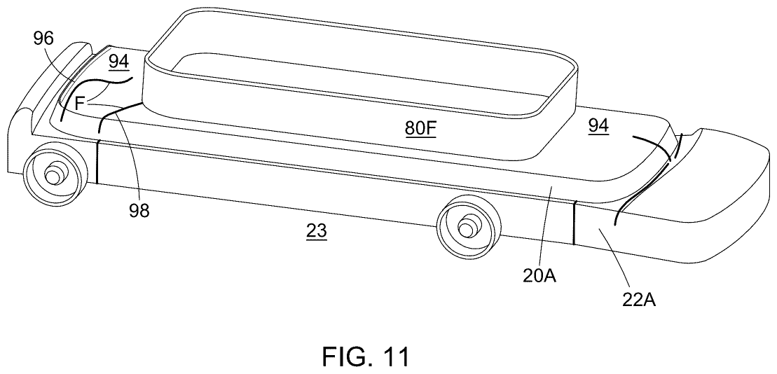

FIG. 1 depicts a radiation treatment system. Typically, such a system is capable of generating either an electron (particle) beam or an x-ray (photon) beam for use in the radiotherapy treatment of patients on a radiotherapy patient positioning system 10. Other radiation treatment systems are capable of generating heavy ion particles such as protons. For purposes of the present discussion, only x-ray irradiation will be discussed. However, it will be appreciated by those skilled in the art that the same principles apply to other systems.

Stand 1 supports a rotatable gantry 2 with a treatment head 3. Next to stand 1 there is arranged a control unit (not shown) that includes control circuitry for controlling the different modes of operation of the accelerator. A high voltage source is provided within the stand or in the gantry, to supply voltage to an electron gun (not shown) positioned on an accelerator guide located in the gantry 2. Electrons are emitted from the electron gun into the guide (not shown) where they are accelerated. A source supplies RF (microwave) power for the generation of an electric field within the waveguide. The electrons emitted from the electron gun are accelerated in the waveguide by the electric field, and exit the waveguide as a high energy electron beam, typically at megavoltage energies. The electron beam then strikes a suitable metal target, emitting high energy x-rays in the forward direction.

FIG. 2 is front elevation of a radiotherapy patient positioning system 10. FIG. 2 is intended to show the approximate location of parts of the patient positioning system 10 but does not show these parts individually in detail. The radiotherapy patient positioning system 10 includes a patent couch 14 (see FIG. 3) having a footrest at location 16, a vertical support at location 18 having a foot at location 20, and a base at location 22 that is in contact with a floor 23. A camera post 25 may be fitted to the patient couch 14. The patient couch 14 may be made from carbon fibre.

The couch 14 is coupled at the footrest end to the vertical support by a pitch roll coupling at location 24 and a longitudinal coupling at location 26. The footrest end of the vertical support is connected to the pitch roll coupling. The pitch roll coupling is connected to the vertical support by the longitudinal coupling. The longitudinal coupling is connected to the vertical support. The foot of the vertical support is connected to the base.

The pitch roll coupling allows movement of the patent couch 14 with respect to the base about a roll axis “R” (see FIG. 3). The pitch roll coupling also allows movement of the patent couch 14 with respect to the base about a pitch axis “P”. The longitudinal coupling allows movement of the patent couch 14 with respect to the base along a longitudinal axis “L”. The pitch roll coupling is configured to slide along the longitudinal coupling in the direction of the longitudinal axis “L”. The vertical support allows the height of the patent couch 14 to vary with respect to the base along a vertical axis “H” that is perpendicular to the longitudinal axis “L”.

The foot of the vertical support is coupled to the base such that the foot can move with respect to the base in a lateral direction “LA” that is perpendicular to the vertical axis “H” and that is perpendicular to the longitudinal axis “L”. The foot 20 of the vertical support 18 is coupled to the base 22 such that the foot 20 can move with respect to the base 22 about a yaw axis “Y”. Movement about the yaw axis “Y” is provided by a yaw module at location 27. Due to the (indirect) coupling of the patient couch 14 to the foot, the movement of the foot of the vertical support with respect to the base allows the patient couch 14 to move in the lateral direction “LA” and about the yaw axis “Y”.

The connections described above provide the patient couch 14 with six movement axes, and so allow a patient to be positioned precisely and accurately. The connections and movements described above are generally known and may be implemented by any suitable arrangement—and are not described in detail herein. Fewer than, or more than, six axes of movement may be provided.

The terms “vertical” and “vertically”, and “horizontal” and “horizontally” used herein should not be taken to require a particular orientation of any part when deployed, but are used to describe relative orientations of parts as shown in the figures.

The radiotherapy patient positioning system 10 is provided with a cover 12 in accordance with an embodiment of the invention, as will be described with reference to FIGS. 3 to 11. The cover 12 comprises a plurality of sections:

A footrest cover section 16A that is configured to cover the footrest 16.

A pitch roll cover section 24A that is configured to cover the pitch roll coupling 24. The pitch roll cover section 24A is located below the footrest cover section 16A.

A longitudinal coupling cover section 26A that is configured to cover the longitudinal coupling 26. The longitudinal coupling cover section 26A is located generally below the pitch roll cover section 24A.

A vertical support cover section 18A that is configured to cover the vertical support 18. The vertical support cover section 18A is located below the longitudinal coupling cover section 26A.

A foot cover section 20A that is configured to cover the foot 20. The foot cover section 20A is located below the vertical support cover section 18A.

A base cover section 22A that is configured to cover the base 22.

A yaw module cover section 27A that is configured to cover the yaw module 27.

The parts of the patient positioning system (e.g. the footrest 16, the pitch roll coupling 24, longitudinal coupling 26, vertical support 18, foot 20, base 22 and/or yaw module 27) are covered by one of more of the sections of the cover 12. The parts of the patient positioning system are moveable relative to one another to provide the desired movement of the patient positioning system to support a patient on the couch 14 in a desired position.

The footrest cover section 16A encases one end of the patient couch 14, at the footrest 16. As best shown in FIG. 6, the footrest cover section 16A includes an upper footrest cover section portion 30 that covers the upper main surface of the footrest 16, a lower footrest cover section portion 32 that partially covers the lower main surface of the footrest 16 and a peripheral footrest cover section portion 34 that extends around the periphery of the footrest 16 that connects the upper footrest cover section portion 30 and the lower footrest cover section portion 32. The lower footrest cover section portion 32 tapers downwardly towards a generally rectangular footrest cover section lower opening 36 in the lower footrest cover section portion 32. The generally rectangular lower footrest cover section opening 36 is sized and located to fit over an upper generally rectangular (in horizontal cross-section) pitch roll cover section portion 38 of the pitch roll cover section 24A. The lower footrest cover section portion 32 includes a lower footrest cover section portion lip 40 that extends downwardly around the lower footrest cover section opening 36, overhanging and partially overlapping the exterior of the upper generally rectangular pitch roll cover section portion 38 on three or more sides. The upper footrest cover section portion 30, lower footrest cover section portion 32, peripheral footrest cover section portion 34 and lower footrest cover section portion lip 40 may be integrally formed.

The lower footrest cover section portion lip 40 and the upper generally rectangular pitch roll cover section portion 38 are sized and shaped such that the lower footrest cover section portion lip 40 can slide over and tilt with respect to the lower footrest cover section portion 38 to accommodate movement of the patient couch 14 about the roll axis “R” and the pitch axis “P”. For example, FIG. 6 shows a roll angle of 3°. The lower footrest cover section portion lip 40 always at least partially overlaps the upper generally rectangular pitch roll cover section portion 38, even at the extremes of permitted movement about the roll axis “R” and the pitch axis “P”—and this prevents, or reduces, dirt or fluid entering the pitch roll coupling at location 24 and/or hoses, cables or other accessories being trapped between the patient couch 14 and the pitch roll coupling at location 24.

The upper generally rectangular pitch roll cover portion 38 of the pitch roll cover section 24A extends around and partially overlaps the exterior of a generally vertically extending middle pitch roll cover portion 42 of the pitch roll cover section 24A—and this prevents, or reduces, dirt or fluid entering the pitch roll coupling located at 24.

As mentioned above, the pitch roll coupling located at 24 is connected to the vertical support located at 18 by the longitudinal coupling located at 26. The pitch roll coupling includes a flange 50 (best seen in FIG. 3) that slides within channels 52 at opposite sides of the longitudinal coupling located at 26, and this allows movement of the pitch roll coupling (and the attached patient couch 14) along the longitudinal direction “L”.

An upper part of each channel 52 is formed by a generally horizontal wall 54 of the longitudinal coupling cover section 26A. The generally horizontal walls 54 of the longitudinal coupling cover section 26A each have a curved distal end 56. A lower pitch roll cover portion 58 of the pitch roll cover section 24A extends from the lower end of the middle pitch roll cover portion 42 of the pitch roll cover section 24A to the flange 50. The lower pitch roll cover portion 58 curves inwardly at curved portions 60 to clear the generally horizontal walls 54 and curved distal ends 56 of the longitudinal coupling cover section 26A and then extends generally vertically towards the flange 50.

Liquid running down the generally vertically extending middle pitch roll cover portion 42 of the pitch roll cover section 24A will drip onto the generally horizontal walls 54 of the longitudinal coupling cover section 26A and may flow outwardly, away from the channels 52 or inwardly towards the channels 52. The end of the vertically extending middle pitch roll cover portion 42, where it meets or lies adjacent to the lower pitch roll cover portion 58, may form a “dripping edge”.

Liquid running down the generally vertically extending middle pitch roll cover portion 42 of the pitch roll cover section 24A that flows inwardly towards the channels 52 may be collected in a bucket or reservoir associated with the flange 50. Liquid running down the generally vertically extending middle pitch roll cover portion 42 of the pitch roll cover section 24A that flows inwardly towards the channels 52 may alternatively, or additionally, reach a main horizontal surface 62 of a main body 64 of the longitudinal coupling cover section 26A, the main horizontal surface 62 lying between the channels 52. The main horizontal surface 62 of the longitudinal coupling cover section 26A may be provided with one or more drain holes to allow the liquid to be drained away from the radiotherapy patient positioning system 10. The main horizontal surface 62 of the longitudinal coupling cover section 26A may be formed of metal.

The longitudinal coupling cover section 26A includes a lower engagement portion 66 that is coupled to the vertical cover section located at 18. The lower engagement portion 66 extends around and partially overlaps the vertical cover section 18—and this prevents, or reduces, dirt or fluid entering the vertical cover section located at 18.

The main body 64 of the longitudinal coupling cover section 26A is fixed to the lower engagement portion 66. The generally horizontal wall 54 of the longitudinal coupling cover section 26A extends outwardly beyond the main body 64 to form protrusions 68. The downwardly facing surface 69 of the protrusions 68 may be concave (see FIG. 8).

An upper peripheral surface 70 of the lower engagement portion 66 extends generally horizontally outwardly beyond the generally horizontal wall 54 (in the lateral direction “LA”). Liquid running down the generally vertically extending middle pitch roll cover portion 42 of the pitch roll cover section 24A that drips onto the generally horizontal walls 54 of the longitudinal coupling cover section 26A and flows outwardly, away from the channels 52 will then flow down a vertical surface 72 of the protrusions 68. The protrusions overhang the main body 64. Fluid flowing down the vertical surface 72, when reaching the corner with the downwardly facing surface 69 then drips onto the upper peripheral surface 70 of the engagement portion 66. The concave shape of the downwardly facing surface 69 of the protrusions 68 prevents or reduces any flow of liquid along the downwardly facing surface 69. The corner of the vertical surface 72 with the downwardly facing surface 69 may form a “dripping edge”. Fluid collection walls 74 may extend upwardly from the outer edges of the upper peripheral surface 70 of the engagement portion 66 to form a bucket or reservoir to collect the liquid.

The vertical cover section 18 includes a plurality (six in this example) of similar, but not identical, vertical cover parts 80A to 80F. Each of the vertical cover parts 80A to 80F is a rectangular hollow section part. That is, each of the vertical cover parts 80A to 80F comprises a four-sided generally vertical wall 81 providing a hollow cuboid space within. The lower engagement portion 66 of the longitudinal coupling cover section 26A is coupled to the uppermost vertical cover part 80A of the vertical cover section 18. The lower engagement portion 66 extends around, overhangs and partially overlaps the uppermost vertical cover part 80A. Each of the vertical cover parts 80B to 80F is smaller in width (in along the axis “LA”) and is smaller in depth (along the axis “L”) than the vertical cover part immediately above, so that a lower one of the vertical cover parts 80B to 80F can slide within (along axis “H) the vertical cover parts 80A to 80E immediately above. In this way, the vertical cover parts 80A to 80F comprise a telescopic side wall structure. The telescopic side wall structure is shaped to fit around parts of the positioning system that are moveable relative to one another and the telescopic side wall structure is configured to expand or contract with relative movement of the parts of the positioning system. Such an arrangement allows the height H of the vertical cover section 18 to vary, and therefore the height of the patient couch 14 to vary.

The side wall structure of each vertical cover part 80A to 80F overlaps the side wall structure of an adjacent vertical cover part 80A to 80F. The amount of overlap is variable to accommodate relative movement of the parts of the positioning system.

Referring, for example, to vertical cover part 80D, a lower edge of its side wall structure includes an inwardly extending flange 82 and an upper edge of its side wall structure includes an outwardly extending flange 84. The other vertical cover parts 80A-C and 80E-F include similar inwardly extending flanges 82 and outwardly extending flanges 84 at the lower and upper edges of their side wall structures, respectively. The inwardly extending flange 84 of vertical cover part 80C and the outwardly extending flange 84 of vertical cover part 80D are configured to engage one another to prevent the side wall structure of vertical cover part 80C and the side wall structure of vertical cover part 80D separating in response to the relative movement of the two parts of the positioning system. The inwardly extending flange 82 and the outwardly extending flange 84 of each adjacent vertical cover part are similarly configured to engage one another to prevent the side wall structures of adjacent vertical cover parts separating. Each vertical cover part always at least partially overlaps the adjacent lower vertical cover part, even at the extremes of permitted movement—and this prevents, or reduces, dirt or fluid entering the vertical cover section 18 and/or hoses, cables or other accessories being trapped between the vertical cover parts 80A to 80F.

The foot cover 20A is fixed to the lowermost vertical cover part 80F of the vertical support located at 18. As mentioned above, the foot located at 20 of the vertical support located at 18 is coupled to the base located at 22 such that the foot can move with respect to the base in a lateral direction “LA” that is perpendicular to the vertical axis “H” and that is perpendicular to the longitudinal axis “L”. The foot of the vertical support is coupled to the base such that the foot can rotate with respect to the base about a yaw axis “Y”. Movement about the yaw axis “Y” is provided by a yaw module located at 27. Due to the (indirect) coupling of the patient couch 14 to the foot, the movement of the foot of the vertical support with respect to the base allows the patient couch 14 to move in the lateral direction “LA” and about the yaw axis “Y”.

As shown in FIG. 10, an upper generally horizontal surface 90 of the base cover 22A extends under the lower distal edge of the foot cover 20A. Upper generally horizontal surface 90 may include an upstanding wall 92 at its innermost edge. Fluid flowing down the foot cover 20A will generally then flow down the vertical surface of the base cover 22A. A small portion of the fluid flowing down the foot cover 20A may then flow along upper generally horizontal surface 90 but the upstanding wall 92 and the small inclination of the upper generally horizontal surface 90 will cause any such fluid to be redirected down the vertical surface of the base cover 22A. The distal edge of the foot cover 20A may form a “dripping edge”.

As shown in FIG. 11, the foot cover 20A may have a generally horizontal surface 94 that is attached to the lowermost vertical cover part 80F of the vertical support located at 18. The generally horizontal surface 94 may include one or more fluid-guiding walls or protrusions 96 to direct the flow of fluid along a desired path. As also shown in FIG. 11, the generally horizontal surface 94 may include one or more channels 98 to direct the flow of fluid along a desired path-e.g. to a particular part of the base cover section 22A. The channels 98 may be formed by tongue and groove connections between parts forming the horizontal surface 94.

The cover 12 prevents, or reduces, dirt or fluid entering the radiotherapy patient positioning system 10 that is inside the cover 12 and also prevents hoses, cables or other accessories being trapped between parts of the radiotherapy patient positioning system 10 that is inside the cover 12. The general path of fluid along the cover 12 is indicated in the figures by letter “F”.

The parts of the cover 12 may be made from plastic or any other suitable material. A robust and rigid material is preferred.

The parts of the cover 12 may have seals provided between them.

The parts of the cover 12 are easy to clean, which provides a hygienic design for the operator and patient.

The parts of the cover 12 do not restrict the usual movement of the radiotherapy patient positioning system 10. For example, the six degrees of movement are not inhibited by the parts of the cover 12.

The parts of the cover 12 may be compatible with the current existing mechanics and systems which are already installed, and may be retro-fitted to already deployed radiotherapy patient positioning systems.

Although an embodiment has been described in which the cover 12 comprises a plurality of sections, it should be understood that each section alone is a cover for a radiotherapy patient positioning system that may cover only a portion of the radiotherapy patient positioning system. Thus, a cover for a radiotherapy patient positioning system may include any one of:
A footrest cover section 16A that is configured to cover the footrest 16.
A pitch roll cover section 24A that is configured to cover the pitch roll coupling 24.
A longitudinal coupling cover section 26A that is configured to cover the longitudinal coupling 26.
A vertical support cover section 18A that is configured to cover the vertical support 18.
A foot cover section 20A that is configured to cover the foot 20.
A base cover section 22A that is configured to cover the base 22.
A yaw module cover section 27A that is configured to cover the yaw module 27.

A cover for a radiotherapy patient positioning system may include any combination of these sections. The vertical support cover section 18A may be particularly suitable for stand-alone use with a radiotherapy patient positioning system, including retro-fitting to an already deployed radiotherapy patient positioning system.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A protective cover for a radiotherapy patient positioning system having at least two parts that are moveable relative to one another, the protective cover comprising:
at least a first cover section configured to cover a first part of the at least two parts of the radiotherapy patient positioning system and to overhang a second part of the at least two parts of the radiotherapy patient positioning system.

2. The protective cover of claim 1, further comprising:
a second cover section configured to cover a second part of the at least two parts, wherein the first cover section is configured to overhang the second cover section.

3. The protective cover of claim 2, wherein a size and a shape of the first cover section and a size and a shape of the second cover section are such that liquid running down the protective cover is guided away from the parts of radiotherapy patient positioning system covered by the first cover section and the second cover section.

4. The protective cover of claim 2, wherein the first cover section is configured to partially overlap the second cover section.

5. The protective cover of claim 4, wherein the first cover section is slidably coupled to the second cover section to accommodate movement between the first part and the second part.

6. The protective cover of claim 2, wherein the first cover section and the second cover section comprise a telescopic side wall structure, wherein the telescopic side wall structure is shaped to fit around the first part and the second part, the first part and the second part are moveable relative to one another and the telescopic side wall structure is configured to expand or contract with the relative movement of the first part and the second part.

7. The protective cover of claim 6, wherein the first cover section has a first side wall structure that overlaps a second side wall structure of the second cover section by a variable amount to accommodate the relative movement of the first part and the second part, wherein a lower edge of the first side wall structure includes an inwardly extending flange and an upper edge of the second side wall structure includes a outwardly extending flange, the inwardly extending flange and the outwardly extending flange being configured to engage one another to prevent the first side wall structure and the second side wall structure separating in response to the relative movement of the first part and the second part.

8. The protective cover of claim 2, wherein the first cover section includes at least one lip that is configured to overhang the second part.

9. The protective cover of claim 8, wherein the at least one lip is configured to partially overlap the second part.

10. The protective cover of claim 8, wherein the at least one lip extends in a first direction along a side of the first cover section and is configured to allow movement of the first part relative to the second part in the first direction.

11. The protective cover of claim 8, wherein a size and a shape of the first cover section is such that liquid running down the protective cover is guided away from the second part.

12. The protective cover of claim 1, wherein the first cover section extends generally vertically and the protective cover further comprises:
a further cover section that extends generally horizontally, the further cover section being provided with one or more drainage holes.

13. The protective cover of claim 12, wherein the first cover section extends generally vertically and the protective cover further comprises:
a further cover section that extends generally horizontally, the further cover section being provided with one or more protrusions for guiding flow of liquid in a desired direction.

14. A radiotherapy patient positioning system covered with the protective cover of claim 1.

15. A radiotherapy patient positioning apparatus including:
a radiotherapy patient positioning system having at least two parts that are moveable relative to one another; and
the protective cover of claim 1.

16. The protective cover of claim 3, wherein the first cover section and the second cover section comprise a telescopic side wall structure, wherein the telescopic side wall structure is shaped to fit around the first part and the second part, the first part and the second part are moveable relative to one another and the telescopic side wall structure is configured to expand or contract with the relative movement of the first part and the second part.

17. The protective cover of claim 4, wherein the first cover section and the second cover section comprise a telescopic side wall structure, wherein the telescopic side wall structure is shaped to fit around the first part and the second part, the first part and the second part are moveable relative to one another and the telescopic side wall structure is configured to expand or contract with the relative movement of the first part and the second part.

18. The protective cover of claim 5, wherein the first cover section and the second cover section comprise a telescopic side wall structure, wherein the telescopic side wall structure is shaped to fit around the first part and the second part, the first part and the second part are moveable relative to one another and the telescopic side wall structure is configured to expand or contract with the relative movement of the first part and the second part.

19. The protective cover of claim 9, wherein a size and a shape of the first cover section is such that liquid running down the protective cover is guided away from the second part.

20. The protective cover of claim 10, wherein a size and a shape of the first cover section is such that liquid running down the protective cover is guided away from the second part.
